(19) United States
(12) Patent Application Publication
(10) Pub. No.: US 2016/0367268 A1
(43) Pub. Date: Dec. 22, 2016

(54) CUSTOMIZABLE SURGICAL TEMPLATE

(71) Applicant: William J. Martin, Lexington, KY (US)

(72) Inventor: William J. Martin, Lexington, KY (US)

(21) Appl. No.: 15/193,825

(22) Filed: Jun. 27, 2016

Related U.S. Application Data
(62) Division of application No. 14/166,579, filed on Jan. 28, 2014.
(60) Provisional application No. 61/771,252, filed on Mar. 1, 2013.

(51) Int. Cl. A61B 17/17; A61B 90/96; A61B 90/90

(52) U.S. Cl. CPC A61B 17/17; A61B 90/90; A61B 90/96

(57) ABSTRACT

Disclosed herein are embodiments of a customizable surgical template and methods for preparing and providing the same. More specifically, the disclosed surgical template coordinates the arrangement of surgical instruments used for a particular surgical procedure in the order preferred by a particular surgeon or other entity.

### CROSS REFERNCE TO RELATED APPLICATION

This application claims the benefit of priority as a divisional of U.S. patent application Ser. No. 14/166,579, filed Jan. 28, 2014, which claims the benefit of priority of U.S. Provisional Patent Application Ser. No. 61/771,252, filed Mar. 1, 2013, the entireties of these aforementioned applications being incorporated herein by reference.

### FIELD OF THE INVENTION

Disclosed herein are embodiments of a customizable surgical template and methods for preparing and providing the same. More specifically, the disclosed surgical template coordinates the arrangement of surgical instruments used for a particular surgical procedure in the order preferred by a particular surgeon or other entity.

### BACKGROUND OF THE INVENTION

Surgical procedures are complex processes which often require the use of multiple surgical instruments in particular orders. Surgical technicians and other medical professionals are often tasked with collecting, sterilizing, and arranging the instruments prior to the start of a surgical procedure to reduce the risk of errors and ensure that the proper instrument is available to a surgeon at the proper time. However, some surgical procedures may be effected in several different ways. For example, one surgeon may prefer to use a certain set of instruments to accomplish a given task and another surgeon may prefer to use a different set of instruments to accomplish the same task. Furthermore, some steps in a surgical procedure may be performed in different orders. For example, one surgeon may prefer to perform steps A, B, C, then D, while another surgeon may perform the same procedure by performing steps A, C, B, then D. This variety in instrument use and order of steps in surgical procedures complicates the process of collecting, sterilizing, and arranging instruments for a surgeon's use. Without prior experience with a particular surgeon's preferred instruments and order of operation in a specific surgical procedure, there is a risk that a technician may prepare the wrong instruments or may arrange the instruments in an order undesirable to the surgeon. Surgical procedures are often time-sensitive operations, making it problematic and potentially harmful to the patient to delay a procedure to obtain a forgotten instrument.

### SUMMARY

Disclosed herein is a customizable surgical template and method for preparing and providing the same. The surgical template is a sheet designed to be used with a specific surgical procedure and includes graphical representations, such as, for example, silhouettes, outlines or images, of surgical instruments specific for that procedure arranged in a predetermined pattern corresponding to the steps in the surgical procedure. For surgical procedures involving implants, the surgical template may include outlines of the implants specific for that procedure as well. The surgical template serves as an aid for the surgeon and supporting medical professionals, as surgical instruments for a specific surgical procedure can be obtained and laid out in order on the surgical template, the location for each instrument indicated by the graphical representation for that instrument, prior to the beginning of the surgery. Preparation of unnecessary instruments would be immediately visible, as there would be no corresponding graphical representation for that instrument on the surgical template. Also, failure to prepare a needed instrument would be immediately visible, as an graphical representation on the template would not have a corresponding instrument placed atop it.

In some embodiments, a customized surgical template comprises a sheet displaying a graphical representation of at least one surgical instrument used in a specific surgical procedure, the graphical representation being arranged in an order of use of the represented surgical instrument in the surgical procedure. In some embodiments, the sheet displays the graphical representation of each of a plurality of surgical instruments used in the specific surgical procedure, the graphical representations being arranged in the order of use of the represented surgical instruments in the surgical procedure. In further embodiments, the customized surgical template is printed from a customized surgical template record. In certain embodiments, the customized surgical template record is customized to include the at least one surgical instrument selected by a user and to be arranged in the order of use selected by the user. In some embodiments, customizing the surgical template record includes defining at least one step and assigning each of the selected at least one surgical instrument to at least one of the at least one step. In further embodiments, the sheet is capable of withstanding at least one of radiation sterilization, chemical sterilization, and heat sterilization. In certain embodiments, the sheet includes a machine-readable code.

In some embodiments, a system comprises a computer including a processor, memory, and an instrumentation database, the instrumentation database including a plurality of surgical instrument records, each surgical instrument record including a graphical representation of a surgical instrument; and a user interface in electronic communication with the instrumentation database; wherein a surgical template record is prepared by assembling user-selected surgical instruments in a user-selected order. In further embodiments, the system further comprises a printer in electronic communication with the server, the printer for printing a surgical template including the graphical representations each user-selected surgical instrument, the graphical representations being arranged in the user-selected order. In certain embodiments, the surgical template is printed on a sterilizable sheet. In some embodiments, the user-selected surgical instruments are selected based on the preferences of one of a health insurance provider and a surgical instrument provider. In further embodiments, the user-selected surgical instruments are selected based on the preferences of a surgeon.

In some embodiments, a method comprises selecting at least one surgical instrument record, the surgical instrument record including a graphical representation of a surgical instrument; and creating a surgical template record including each selected surgical instrument record arranged in a defined order. In further embodiments, the method further comprises printing a surgical template including the graphical representation of the surgical instrument in each selected surgical instrument record. In certain embodiments, the surgical template is printed on a sterilizable sheet. In some embodiments, each selected surgical instrument record is selected from an instrumentation database including a plurality of surgical instrument records. In further embodiments, creating the surgical template record includes defining at least one step and assigning each selected surgical instrument record to one of the at least one step. In certain embodiments, selecting at least one surgical instrument record is performed by a user and wherein assigning each selected surgical instrument is performed by a user.

In some embodiments, a kit comprises a sterilizable container including a customized surgical template comprising a sterilizable sheet displaying a graphical representation of at least one surgical instrument for use in a specific surgical procedure; and the at least one surgical instrument. In further embodiments, the sheet displays graphical representations of a plurality of surgical instruments for use in the specific surgical procedure, the graphical representations being arranged in the order of use of the plurality of surgical instruments in the surgical procedure, and wherein the sterilizable container includes the plurality of surgical instruments.

It will be appreciated that the various surgical templates and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

A better understanding of the present invention will be had upon reference to the following description in conjunction with the accompanying drawings.

FIG. 1 depicts a first embodiment of a surgical template;

FIG. 2 depicts a second embodiment of a surgical template;

FIG. 3 depicts a third embodiment of a surgical template;

FIG. 4 depicts a fourth embodiment of a surgical template;

FIG. 5 depicts a fifth embodiment of a surgical template; and

FIGS. 6A-6C are flowcharts illustrating an embodiment of a method for making and using a customizable surgical template.

### DETAILED DESCRIPTION OF THE PREFERRED EMBODIMENTS

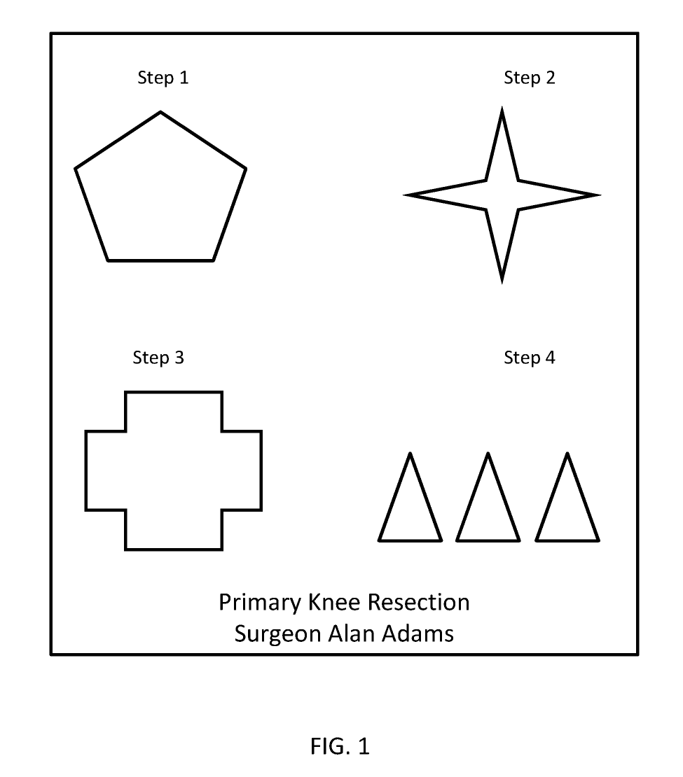

FIGS. 1-3 depict embodiments of surgical templates designed for use with a specific surgical procedure, namely, a primary knee resection. For ease of explanation, the procedure is simplified to include only four steps: preparation and resection of the patella, preparation and resection of the tibia, preparation and resection of the femur, and insertion of prosthetics in each of the patella, tibia, and femur. Each of the steps of preparation and resection of the patella, tibia, and femur may be completed in any order, with the step of inserting prosthetics necessarily occurring after the other three steps. For ease of explanation, each step is accomplished using a different surgical instrument, namely, a pentagon-shaped instrument for preparation and resection of the patella, a four-sided star shaped instrument for preparation and resection of the tibia, a plus-sign shaped instrument for preparation and resection of the tibia, and three triangle-shaped implants for the insertion step. Most actual surgical procedures require multiple steps, and each step may require the use of a plurality of different surgical instruments, each of which would be represented on a surgical template designed for such procedure.

FIG. 1 depicts a first embodiment of a surgical template designed for use with the simplified primary knee resection surgical procedure. This template is designed for a surgeon who prefers to enact the steps of the procedure in the order of (1) preparation and resection of the patella, (2) preparation and resection of the tibia, (3) preparation and resection of the femur, and (4) insertion of implants in each of the patella, tibia, and femur. The surgical template includes graphical representations of each of the surgical instruments used in the procedure. In this embodiment, the graphical representations are outlines of the instruments to be used. Preferably, the surgical template includes identifying information indicating the procedure it is designed for and the surgeon who will carry out the procedure.

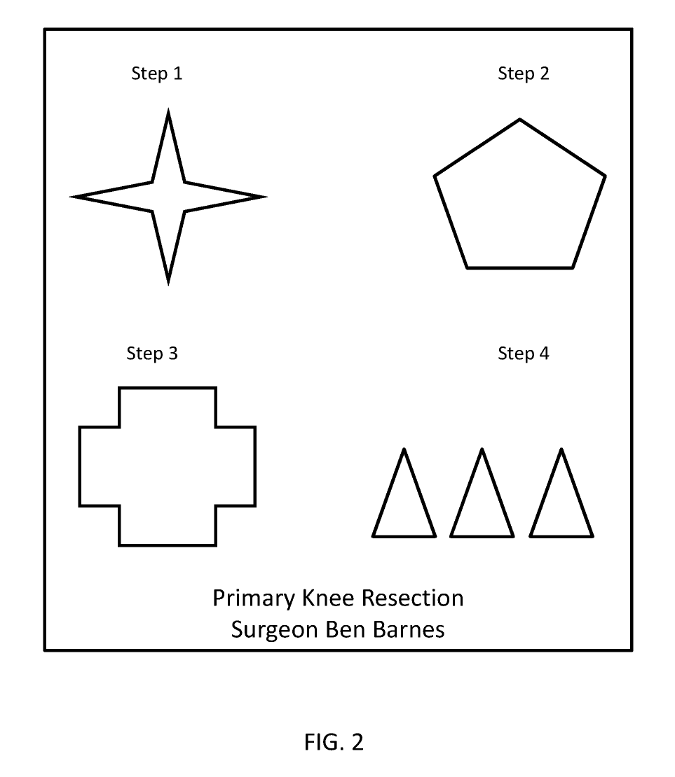

FIG. 2 depicts a second embodiment of a surgical template designed for use with the simplified primary knee resection surgical procedure. This template is designed for a surgeon who prefers to enact the steps of the procedure in the order of (1) preparation and resection of the tibia, (2) preparation and resection of the patella, (3) preparation and resection of the femur, and (4) insertion of implants in each of the patella, tibia, and femur.

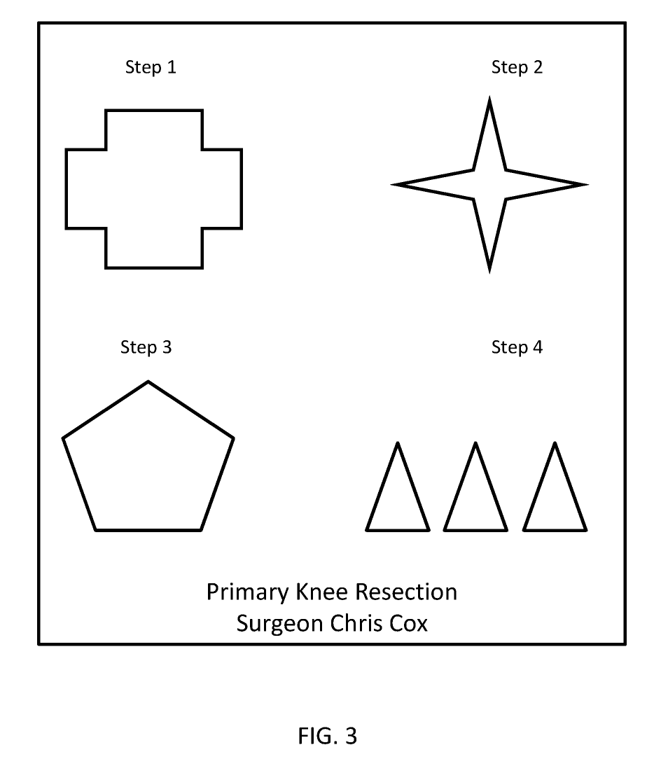

FIG. 3 depicts a third embodiment of a surgical template designed for use with the simplified primary knee resection surgical procedure. This template is designed for a surgeon who prefers to enact the steps of the procedure in the order of (1) preparation and resection of the femur, (2) preparation and resection of the tibia, (3) preparation and resection of the patella, and (4) insertion of implants in each of the patella, tibia, and femur.

In some embodiments, a surgical template may include additional text, collectively referred to as identifying information. In certain embodiments, the surgical template may include a brief summary of the tasks the surgeon must perform during each step of the surgical procedure. In further embodiments, the surgical template may include the name of each surgical instrument printed near the graphical representation of that instrument. In some embodiments, the surgical template may include identifying information including, but not limited to, one or more of the following: the name of the surgical procedure the surgical template is designed for, the name of the surgeon the surgical template is customized for, the name of the entity who designed the surgical template, and, for each step, a summary of the task(s) to be performed during that step.

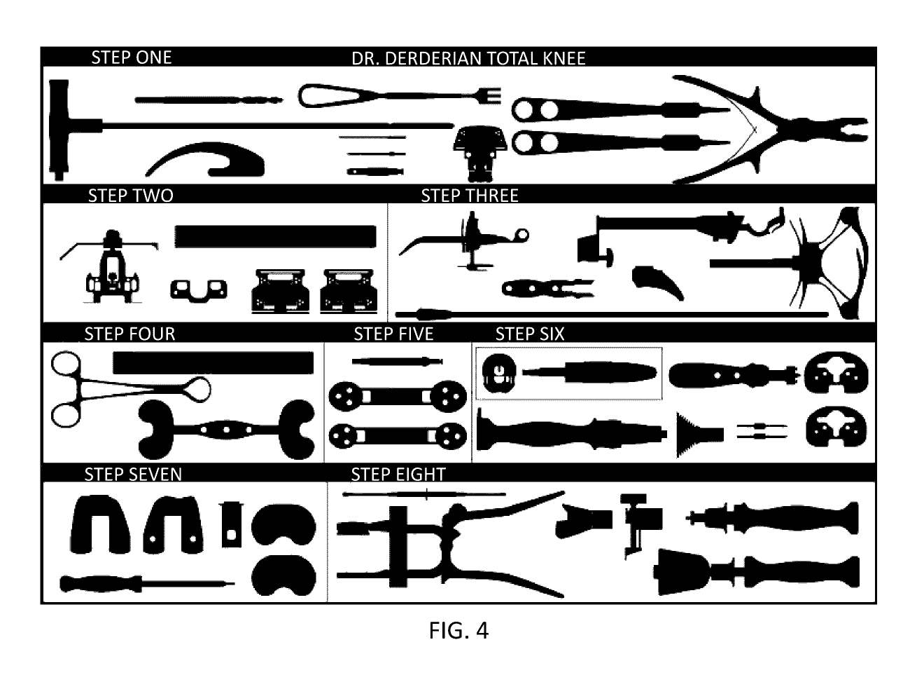

FIG. 4 depicts a fourth embodiment of a surgical template designed for use with a total knee replacement surgical procedure. This depicted embodiment includes identifying information, including the name of the surgeon (Dr. Derderian) and the name of the procedure (Total Knee) at the top of the template. The template is divided into sections, identified as “Step One,” “Step Two,” up to “Step Eight.” Each section includes preferably life-sized graphical representations of the instruments and implants to be used at that step of the procedure. In this embodiment, the graphical representations of the instruments are silhouettes of the instruments and implants. Each graphical representation is accompanied by its own identifying information, namely, the name of the depicted instrument.

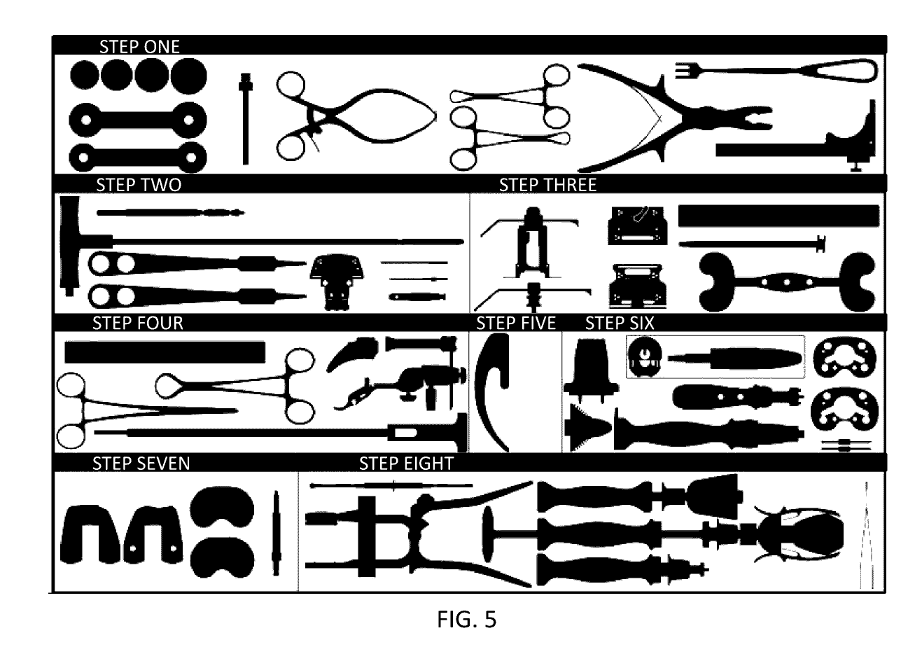

FIG. 5 depicts a fifth embodiment of a surgical template. As in the fourth embodiment, this embodiment of the template is divided into sections, identified as “Step One,” “Step Two,” up to “Step Eight.” Each section includes preferably life-sized graphical representations of the instruments and implants to be used at that step of the procedure. In this embodiment, the graphical representations of the instruments and implants are silhouettes of the instruments and implants. Each graphical representation is accompanied by its own identifying information, namely, the name of the depicted instrument or implant. This embodiment lacks identifying information listing the name of the procedure the surgical template is designed for. This embodiment may be used, among other purposes, as a teaching tool. For example, surgeons, medical students, medical professionals, medical device salespersons, or other individuals may be required to identify a surgical procedure based on the instruments used and the order of use.

A surgical template may be customized by a surgeon, a medical professional acting on behalf of a surgeon or on his or her own behalf, a health insurance provider, a supplier of surgical instruments, such as a vendor or manufacturer or salesperson, an educational institution, a medical services provider, such as a hospital or clinic, or other entity with an interest in surgical procedures. For example, a specific step in a given procedure may be accomplished using one of three different surgical instruments, each provided by a different vendor. A surgical template could be customized by a surgical instrument vendor to include the surgical instrument sold by that vendor. The vendor could then offer the vendor-designed surgical template to medical professionals to encourage sales of the vendor's surgical instruments. For another example, a specific step in a given surgical procedure may be accomplished using one of three different disposable surgical instruments, one of which is significantly less expensive than the others. A surgical template could be customized by an insurance company to include the less expensive disposable surgical instrument to minimize the cost of the procedure. The insurance company could then require that surgeons seeking compensation from the insurance company only perform that surgical procedure using the instruments and order laid out in the surgical template designed by the insurance company. For a further example, a specific step in a given procedure may be accomplished using one of three different surgical instruments, one of which is more comfortable than the others for operation by a left-handed surgeon. A surgical template could be customized by a left-handed surgeon or medical professionals supporting a left-handed surgeon to include the more comfortable instrument. For an additional example, a medical student, surgical technician, or medical device salesperson could be tasked with creating a surgical template for a given surgical procedure as a test of whether that individual had memorized the steps to perform the procedure and the surgical instruments needed for each step.

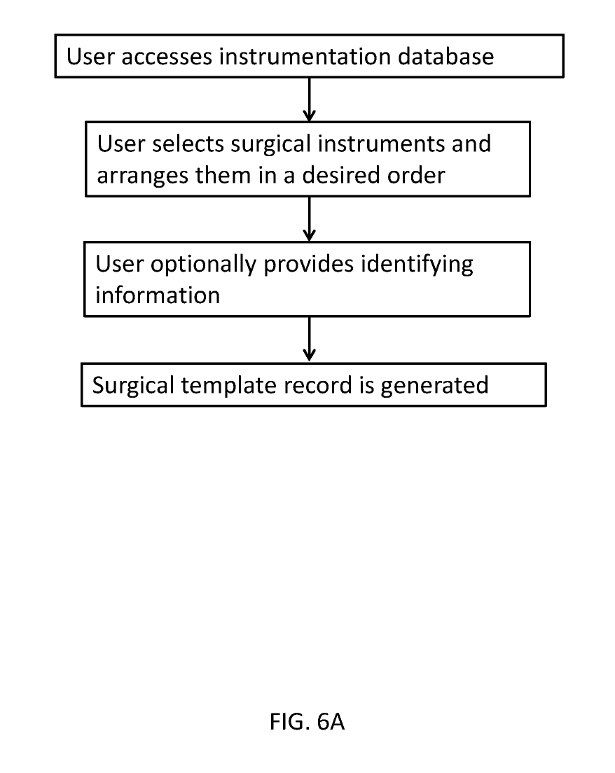

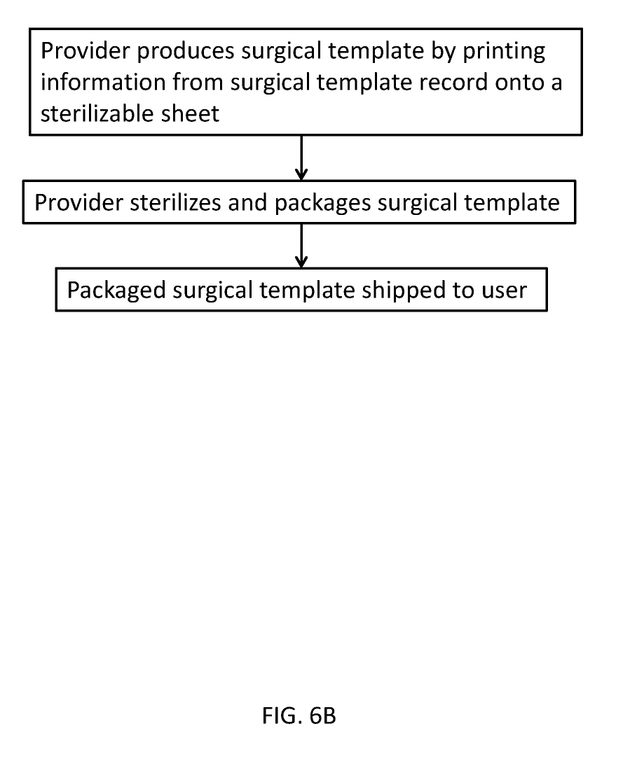

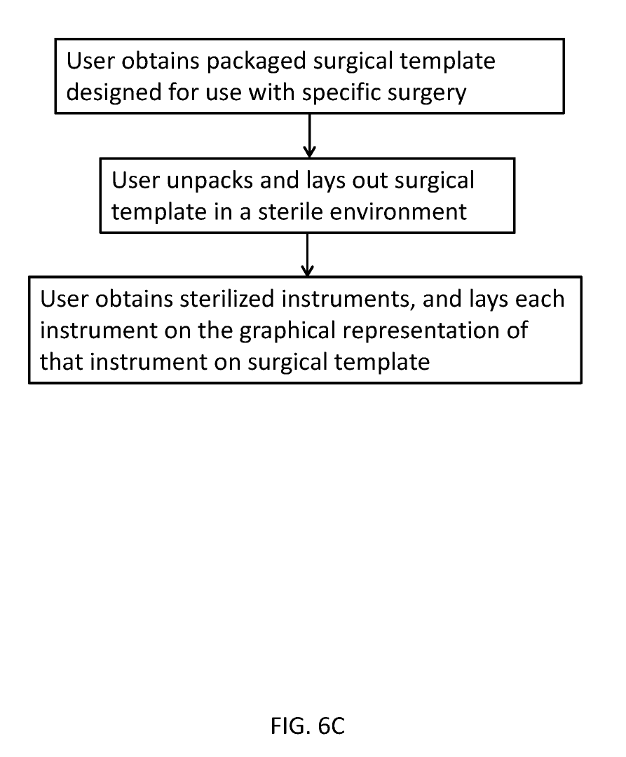

FIGS. 6A, 6B, and 6C are flowcharts illustrating an embodiment of a method for preparing and using customizable surgical templates. In some embodiments, a provider of surgical templates maintains an instrumentation database. In certain embodiments, the instrumentation database is a computer database maintained on a computer (which may be used as a server, client, resource, interface components and the like). A computer utilized in conjunction with embodiments described herein will typically include a processor in communication with memory, and a network interface. Power, ground, clock, and other signals and circuitry are not discussed, but will be generally understood and easily implemented by those skilled in the art. The processor in some embodiments is a microcontroller or general purpose microprocessor that reads its program from memory. The memory in various embodiments includes one or more types such as solid-state memory, magnetic memory, optical memory, or other computer-readable, non-transient storage media. The network interface connects the computer to a data network for electronic communication of data between the computer and other devices attached to the network.

The instrumentation database is accessible by a user remotely accessing the computer, such as, for example, by using a user-interface to access an internet application in electronic communication with the computer. The instrumentation database includes records for surgical instruments. As used herein, the term “surgical instruments” refers to surgical tools, equipment, implants, consumables, such as medical tapes, gauze, sutures, pins, or screws, and other materials which may be used in a surgical procedure. Each surgical instrument record includes at least the name of the instrument and a graphical representation of the surgical instrument, such as an outline, silhouette, or image of the instrument. In some embodiments, a surgical instrument record further includes a list of surgical procedures that instrument has been used with. In certain embodiments, a surgical instrument record further includes the vendor or supplier of that instrument. Surgical instrument records may be added to the instrumentation database as new instruments are developed and may be removed as instruments become obsolete or are no longer used.

In some embodiments, as shown in FIG. 6A, the user may create a surgical template record by selecting at least one surgical instrument from the instrumentation database and defining an order of use for the selected surgical instrument(s). In some embodiments, defining the order of use includes defining at least one step and assigning each selected surgical instrument to a step. In other embodiments, defining the order of use includes placing selected instruments in sequential order. Alternatively, the user may select a pre-made surgical template for a given surgical procedure and change the order of steps in the template or replace pre-selected surgical instruments with preferred alternative surgical instruments in the instrumentation database.

The user may optionally provide identifying information, including, but not limited to, the name of the surgeon for whom the surgical template is being prepared, the name of the surgical procedure, the name of the entity who designed the surgical template, or notes describing the template or surgical procedure. After the user has finished selecting and ordering surgical instruments and providing identifying information for the surgical template, the compilation is stored as a surgical template record. In certain embodiments, the instrumentation database includes pre-made surgical template records for specific surgical procedures, which the user may modify instead of creating a surgical template record from scratch. In some embodiments, pre-made and user-created surgical template records are available for viewing and use by users in a common pool. In other embodiments, where privacy or trade secrets are to be protected, certain surgical templates may only be made available to a subset of the users.

In some embodiments, the provider maintains a computer system including the instrumentation database, an internet application coupled to the instrumentation database, and a database of user accounts. Preferably, the computer system maintains electronic surgical template records storing each surgical template designed by a particular user. A user may log onto the internet application by using a user interface, such as, for example, by accessing a website associated with the application. In some embodiments, each user is associated with a user account and logging onto the application requires the user to prove a password associated with that user account. Once logged on, the user may create new surgical templates by accessing the instrumentation database, access previously generated surgical template records, whether user-created or pre-made by the provider or others, and order one or more surgical templates based on a selected surgical template record.

Once an order has been made, the provider creates one or more surgical templates based on the user-selected surgical template record. In some embodiments, as shown in FIG. 6B, the provider prints the information stored in the surgical template record onto a sheet, such as a sheet of paper, to create the surgical template. The surgical template is constructed using a sheet of paper capable of being sterilized. In some embodiments, the number and size of the surgical instruments to be represented on the template will require that the surgical template be constructed using wide-format paper. In some embodiments, the surgical template is printed using a wide format printer, such as, for example a Hewlett-Packard DesignJet T790 or T2300 eMFP, a Rowe RS-series digital printer, or other suitable wide format printer. For particularly lengthy or equipment-intensive surgical procedures, the surgical template may be printed on multiple sheets.

In preferred embodiments, the surgical template is constructed of a sheet capable of withstanding at least one of radiation sterilization, chemical sterilization, and heat sterilization without significant degradation of the paper or graphical representations or identifying information printed thereon. In some embodiments, the surgical template is constructed of a sheet capable of withstanding sterilization by exposure to gamma radiation, such as, for example, bond paper. In some embodiments, the surgical template is printed onto 20 lb. wide-format bond paper. In other embodiments, the surgical template is printed onto 18 lb. wide-format translucent bond paper. In some embodiments, the surgical template is printed using a pigment-based ink that provides a surface treatment, but does not penetrate the paper. Once the ink has dried, the paper may be sterilized by gamma radiation or other suitable means for sterilization. In certain embodiments, information stored in the surgical template record is printed onto a sheet of paper, which is then laminated. In other embodiments, information stored in the surgical template record is printed onto a sheet of thin plastic, such as polyolefin plastic paper. In some embodiments, the surgical template is printed, sterilized, then sealed in a package with a sterile interior. The surgical template may be rolled or folded prior to packing to decrease the size of the package. The package is then delivered to the user.

In further embodiments, other methods for creating surgical templates may be utilized. In certain embodiments, users with access to suitable printing facilities may access surgical template records and print their own surgical templates locally instead of having them created and delivered by the provider. In further embodiments, the provider managing the computer system may engage a supplier to produce and deliver surgical templates based on surgical template records.

A medical services provider, such as a hospital, may maintain a supply of surgical templates for various surgical procedures. For each given surgical procedure, the medical services provider may maintain multiple surgical templates, each customized to the preferences of different surgeons. In some embodiments, as shown in FIG. 6C, a user, such as a medical technician, may obtain a surgical template designed for use with an upcoming surgical procedure and customized to the preferences of the surgeon who will be performing the procedure. The user will then unpack the surgical template from its sterile packaging in a sterile environment, such as an operating room. The user will lay the surgical template out on a flat surface, such as a sterile tabletop, then obtain pre-sterilized surgical instruments corresponding to each graphical representation on the surgical template. The user will then lay the surgical instruments on the surgical template to ensure that each instrument is present and arranged in the surgeon's preferred order prior to the beginning of the surgery.

In some embodiments, a surgical template may be used to ensure that desired surgical instruments are properly sterilized prior to use. In these embodiments, a user, such as a medical technician, may obtain a surgical template designed for use with an upcoming surgical procedure and customized to the preferences of the surgeon who will be performing the procedure. The user will then unpack the surgical template from its sterile packaging in a non-sterile environment. The user will lay the surgical template out on a flat surface, such as a tabletop, then obtain non-sterile surgical instruments corresponding to each outline on the surgical template. The user will then lay the surgical instruments on the surgical template to ensure that each instrument is present. The user will then sterilize the surgical template and surgical instruments, either separately or together.

In some embodiments, the surgical template is discarded after use. In other embodiments, the surgical template is cleaned after use. In still other embodiments, the surgical template is cleaned, sterilized, and repacked in a sterile packaging after use. When a user's supply of surgical templates is low, the medical services provider or other user can order additional packaged surgical templates from the provider.

In some embodiments, a QR code, bar code, or similar machine-readable code is included on the surgical template. The code can be added by the user or the system provider for inventory tracking purposes so that individual templates can be identified. The code can be included on the surgical template at the time of printing and be associated with the surgical template record to record how many times a particular template is printed and which user printed the template. In other embodiments, the code can be included on the surgical template after printing, such as, for example, by attaching to the template a sterilizable sticker displaying the code. In some embodiments, the machine-readable code, when read, provides data including, but not limited to, at least one of the following: the name of the surgeon for whom the surgical template is designed, a product code for the surgical template, the name of the surgical procedure for which the surgical template is designed, and the price of the surgical template.

In some embodiments, a kit includes a sterilizable container including a surgical template displaying a graphical representation of at least one surgical instrument for use in a specific surgical procedure and the at least one surgical instrument used in that surgical procedure. In some embodiments, the container is a tray with a lid and a plurality of holes or vents. The holes or vents are sized to allow atmospheric communication between the exterior and interior of the tray, while retaining instruments and the surgical template within the tray. A medical technician, upon receiving instructions that a specific surgical procedure will be performed by a specific surgeon, can obtain a pre-packaged kit including the surgical template and necessary instruments, implants, and consumables for that procedure. The technician may then place the entire container within an autoclave to sterilize the surgical template, instruments, implants, and consumables in a single step. In other embodiments, other sterilization methods may be used.

The foregoing detailed description is given primarily for clearness of understanding and no unnecessary limitations are to be understood therefrom for modifications can be made by those skilled in the art upon reading this disclosure and may be made without departing from the spirit of the invention.

What is claimed is:

1) A system comprising:
a computer including a processor, memory, and an instrumentation database, the instrumentation database including a plurality of surgical instrument records, each surgical instrument record including a graphical representation of a surgical instrument; and
a user interface in electronic communication with the instrumentation database;
wherein a surgical template record is prepared by selecting at least one surgical instrument from the instrumentation database and defining an order of use for the selected at least one surgical instrument.

2) The system of claim 1, further comprising a printer in electronic communication with the computer, the printer for printing a surgical template derived from the surgical template record, the surgical template including the graphical representations each selected surgical instrument, the graphical representations being arranged in the defined order of use.

3) The system of claim 2, wherein the surgical template is printed on a sterilizable sheet.

4) The system of claim 2, wherein the surgical template includes a machine-readable code.

5) The system of claim 1, wherein the selected surgical instruments are selected based on the preferences of one of a health insurance provider, a surgical instrument provider, a surgeon, a medical professional, a medical services provider, and an educational institution.

6) The system of claim 1, wherein the order of use is defined based on the preferences of one of a health insurance provider, a surgical instrument provider, a surgeon, a medical professional, a medical services provider, and an educational institution.

7) The system of claim 1, wherein the selected at least one surgical instrument is a plurality of surgical instruments.

8) The system of claim 1, wherein defining the order of use includes defining at least one step and assigning each selected surgical instrument to one of the at least one steps.

9) A method comprising:
selecting at least one electronic surgical instrument record, the surgical instrument record including a graphical representation of a surgical instrument; and
creating an electronic surgical template record including each selected surgical instrument record arranged in a defined order.

10) The method of claim 9, further comprising printing a surgical template including the graphical representation of the surgical instrument in each selected surgical instrument record arranged in the defined order.

11) The method of claim 10, wherein the surgical template is printed on a sterilizable sheet.

12) The method of claim 10, further comprising sterilizing the sheet then sealing the sheet in a package having a sterile interior.

13) The method of claim 10, wherein said sheet is laminated after being printed.

14) The method of claim 9, wherein each selected surgical instrument record is selected from an instrumentation database including a plurality of surgical instrument records.

15) The method of claim 9, wherein creating the electronic surgical template record includes defining at least one step and assigning each selected surgical instrument record to one of the at least one step.

16) The method of claim 15, wherein creating the electronic surgical template record includes defining a plurality of steps and assigning each selected surgical instrument to at least one of the plurality of steps.

17) The method of claim 15, wherein selecting at least one electronic surgical instrument record is performed by a user and wherein assigning each selected surgical instrument is performed by a user.
